(19) United States
(12) Patent Application Publication
(10) Pub. No.: US 2005/0037039 A1
(43) Pub. Date: Feb. 17, 2005

(54) Composition for treatment of tinea pedis and method of use

(76) Inventor: William M. Yarbrough, Peoria, IL (US)

Correspondence Address: Robert L. Knechtel, 1105 Moraing, Woodstock, IL, 60098, US

(21) Appl. No.: 10/638,438

(22) Filed: Aug. 12, 2003

(51) Int. Cl. A61K 9/00; A61K 6/00

(52) U.S. Cl. 424/401

(57) ABSTRACT

A treatment for tenia pedis is provided for in a topical treatment. According to the invention, a method is provided for applying composition substances to the affected area, working the composition into the affected area, and removing the composition from the affected area. The composition comprises at least one ethoxylate and a sarcosinate. Alternatively, the ethoxylate can be exchanged for a methoxylate or a propoxylate. Acetylated lanolin alcohol, sodium lauroyl sarcosinate, EDTA, a foam stabilizer, a scrubbing agent, and water can also be added to the composition to assist performance.

### I. FIELD OF THE INVENTION

The present invention relates to a composition including a sarcosinate and an ethoxylate and a method for using the same in the treatment of tinea pedis infections.

### II. BACKGROUND OF THE INVENTION AND PRIOR ART

Tinea pedis, or athlete's foot, is a common, persistent foot infection. It is a fungal infection caused by one of four dermatophytes, the most common being trichophyton rubrum. It is a microscopic fungus that lives on dead tissue of the hair, toenails, and outer skin layers. The fungus thrives in warm, moist environments including shoes, socks, and the floors of public showers, locker rooms, and swimming pools. Athlete's foot is transmitted through contact with a cut or abrasion on the plantar surface (bottom) of the foot. The infection causes raised, circular pimples or blisters.

There are four common forms of athlete's foot. The most common is a persistent itching of the skin on the sole of the foot or between the toes. As the infection progresses, the skin grows soft. The center of the infection is inflamed and sensitive to the touch. Gradually, the edges of the infected area become milky white and the skin begins to peel. There may also be a slight watery discharge.

The second presentation is the ulcerative type. In this presentation, the peeling skin becomes worse. Large cracks develop in the skin, making the patient susceptible to secondary bacterial infections. The infection can be transmitted to other parts of the body by scratching, or contamination of clothing or bedding.

The third type of infection is often called “moccasin foot.” In this type, a red rash spreads across the lower portion of the foot in the pattern of a moccasin. The skin in this region gradually becomes dense, white, and scaly.

Finally, the fourth form of tinea pedis is inflammatory or vesicular, in which a series of raised bumps or ridges develops under the skin on the bottom of the foot, typically in the region of the metatarsal heads. Itching is intense and there is less peeling of the skin.

People with acute tinea infections may develop similar outbreaks on their hands, typically on the palms. This trichophyde reaction, also known as tineas manuum, is an immune system response to fungal antigens (antibodies that fight the fungal infection).

Tinea has proven difficult to eliminate and often recur. Infections may disappear spontaneously or persist for years. Best results usually are obtained with early treatment before the fungal infection establishes itself firmly.

Historically, antifungal drugs have been used. For early “mild” cases, Imidazole class drugs are used to combat fungal infections by attacking the enzymes of the fungal cell walls, inhibiting growth and reproduction. Examples of these drugs include clotrimazole (Lotrimin®) and miconazole (contained in Lotrimin® and Absorbine Jr.@). They are applied topically and massaged into the skin. They must be reapplied every few hours over a period of weeks.

More difficult cases may require the use of Allylamines class drugs. These drugs include terbinafine (contained in Lamisil®) and naftifine (Naftin®) and are available in prescription form.

The most difficult cases must be treated with drugs such as griseofulvin (Fulvicin® and Grisactin®) and concentrated forms of terbinafine and itraconazole. Griseofulvin can cause side effects such as headache, nausea, and numbness, so it is used as a last resort.

These treatments are not without shortcomings. The topical treatments require use for many days, if not weeks. They are messy creams and lotions. The internal drug can cause side effects, including headaches, nausea, and in extreme cases, organ damage. There is need for, therefore, for a treatment for tinea pedis that is sake, effective, and easy to use. The present invention meets this need. The Inventor has discovered that a combination of a sarcosinate and an ethoxylate can provide relief in many cases of tenia pedis.

### III. OBJECTS OF THE INVENTION

It is an object of the present invention to provide a treatment that helps to alleviate tenia pedis.

It is a further object of the present invention to provide a method of use of the present inventive treatment.

It is yet another object of the present invention to provide a treatment that includes at least an ethoxylate and a sarcosinate.

It is a yet further object of the present invention to provide a treatment that is safe to use.

It is yet another object of the present invention to provide such treatment that is topical, can be purchased over the counter, and is economical.

### IV. SUMMARY OF THE INVENTION

The above objects of the invention are provided for in a topical treatment for tenia pedis. According to the invention, a method is provided for applying a composition of substances to the affected area, working the composition into the affected area, and removing the composition from the affected area. The composition comprises at least one ethoxylate in combination with a sarcosinate. Additionally, acetylated lanolin alcohol, a second ethoxylate, EDTA, a foam stabilizer, an inert scrubbing agent, such as polyethylene beads, and water can also be added to the composition without effecting performance.

Other formulas that keep the polarity similar to that of the inventive formula will also work. To keep the polarity similar, it is necessary for the compound to have similar characteristics, such as Carbon chains, carbonyl groups, Nitrogen bound to Carbon, Aromatic ring(s), Oxylate groups, and appropriate functional groups at the ends of the individual molecules. The ideal substitute chemicals would have all of the characteristics mentioned above, but it is not necessary to have every one of those as listed. For example, if the functional groups at the ends of the individual molecules are exchanged for other functional groups that retain the ability to undergo an emulsion polymerization, then the effectiveness of the compound is also retained. Another example is to change the ethoxylate to a methoxylate or propoxylate. These formations would still retain a similar polarity but would be different compounds with different characteristics. Yet another example would be to exchange triply bound Nitrogen with a doubly bound or perhaps Nitrogen with 4 Carbons bound to it.

### V. DETAILED DESCRIPTION OF THE INVENTION

Chemical analysis and research has revealed that the inventive composition including at least an ethoxylate and a sarcosinate is effective in the treatment of tinea pedis. The Inventor has found that a nonyl phenol ethoxylate and sodium lauroyl sarcosinate work best in combination.

The Inventor has also found, however, that other formulas that keep the polarity similar to that of the inventive formula will also work. To keep the polarity similar, it is necessary for the compound to have similar characteristics, such as Carbon chains, carbonyl groups, Nitrogen bound to Carbon, Aromatic ring(s), Oxylate groups, and appropriate functional groups at the ends of the individual molecules. The ideal substitute chemicals would have all of the characteristics mentioned above, but it is not necessary to have every one of those as listed. For example, if the functional groups at the ends of the individual molecules are exchanged for other functional groups that retain the ability to undergo an emulsion polymerization, then the effectiveness of the compound is also retained. Another example is to change the ethoxylate to a methoxylate or propoxylate. These formations would still retain a similar polarity but would be different compounds with different characteristics. Yet another example would be to exchange triply bound Nitrogen with a doubly bound or perhaps Nitrogen with four carbons bound to it.

A scrubbing agent can also be used scrubbing agent, which preferably is polyethylene granule beads. They should be large enough to be effective but not so large as to cause abrasions. The inventor suggests beads in the range of 5 to 50 microns with an average size being approximately 25 microns or 50 mesh.

To make the inventive composition, an exact ratio of ethoxylate to sarcosinate (in the following discussion sodium lauroyl sarcosinate is used but any sarcosinate meeting the requirements can be used) is not critical. The only requirement is that the ethoxylate is completely reacted with the SLS, creating a polymer. This will vary with the ethoxylate used, but the Inventor has determined that a ratio of ethoxylate-to-SLS of 1.5:2 is preferred. The amount by weight of polyethylene beads can vary according to the grittiness desired. The Inventor has found that a formula of ethoxylate:SLS:polyethylene of 40:20:40 is preferred but that formulas of other concentrations are useful. Thus, for production purposes, formulas having SLS ranging from 10 to 20% by weight, ethoxylate ranging from 20 to 40% by weight, and polyethylene beads from 20 to 50% by weight are reasonable. But again, the formula is not restricted to these ranges, which ranges are presented for example purposes only.

Also, a cutting agent that does not chemically react with the composition may be added. The cutting agent makes the overall composition flow more easily, thereby enabling more packaging options, such as tubes. The cutting agent must be added only in sufficient amount that it promotes flow but does not effect the action of the composition.

In use, a sufficient amount of the composition is used to cover the affected area, the composition is applied to an affected area and worked over the area by a scrubbing motion. After sufficient time has elapsed to ensure that the affected area has been adequately exposed to the composition such that they area feels clean, approximately ten to thirty seconds for the typical person, the area is rinsed cleaned.

1. A treatment for tenia pedis comprising a sarcosinate and an ethoxylate.

2. The treatment for tenia pedis of claim 1 wherein the sarcosinate is sodium lauroyl sarcosinate.

3. The treatment for tenia pedis of claim 1 wherein the ethoxylate is a nonyl phenyl ethoxylate.

4. The treatment for tenia pedis of claim 1 further including a scrubbing agent.

5. The scrubbing agent of claim 4 being polyethylene granules.

6. The polyethylene granules of claim 5 in which the granules are in the range of 4 to 50 microns.

7. The treatment for tenia pedis of claim 1 further including a second nonyl phenyl ethoxylate.

8. The treatment for tenia pedis of claim 1 further including acetylated lanolin alcohol.

9. The treatment for tenia pedis of claim 1 further including water.

10. The treatment for tenia pedis of claim 1 further including ethylenediaminetetraacetc acid.

11. The treatment for tenia pedis of claim 1 further including a foam stabilizing agent.

12. The treatment for tenia pedis of claim 1 further including a cutting agent.

13. The cutting agent of claim 12 being selected from the group of aqueous based solutions and oil based solutions.

14. The treatment for tenia pedis of claim 1 wherein the ethoxylate is exchanged for a methoxylate.

15. The treatment for tenia pedis of claim 1 wherein the ethoxylate is exchanged for a propoxylate.

16. A treatment for tenia pedis comprising: a first ethoxylate, acetylated lanolin alcohol, sodium lauroyl sarcosinate, EDTA, a foam stabilizer, water, and inert polyethylene granules.

17. The treatment for tenia pedis of claim 16 wherein the first ethoxylate is exchanged for a methoxylate.

18. The treatment for tenia pedis of claim 16 wherein the first ethoxylate is exchanged for a propoxylate.

19. A treatment for tenia pedis comprising an ethoxylate, sodium lauroyl sarcosinate, and EDTA.

20. The treatment for tenia pedis of claim 19 wherein the ethoxylate is exchanged for a methoxylate.

21. The treatment for tenia pedis of claim 19 wherein the ethoxylate is exchanged for a propoxylate.

22. A method for treating tenia pedis comprising the steps of:
preparing a composition comprising an ethoxylate and a sarcosinate;
applying the composition to an affected area;
permitting the composition to remain on the affected area a sufficient amount of time to enable the composition of matter to cause an effect; and, removing the composition from the affected area.

23. The method of claim 22 wherein preparing the composition further includes adding second ethoxylate.

24. The method of claim 22 wherein preparing the composition further includes adding acetylated lanolin alcohol.

24. The method of claim 22 wherein preparing the composition further includes adding a scrubbing agent.

25. The scrubbing agent of claim 24 being polyethylene granules.

26. The method of claim 22 wherein preparing the composition further includes adding water.

27. The method of claim 22 wherein preparing the composition further includes EDTA.

28. The method of claim 22 wherein preparing the composition further includes a foam stabilizer.

29. The method of claim 22 further including the step of adding a thinning agent to the composition.

30. The method of claim 22 wherein the ethoxylate is exchanged for a methoxylate.

31. The method of claim 22 wherein the ethoxylate is exchanged for a propoxylate.
